(19) United States
(12) Patent Application Publication
(10) Pub. No.: US 2025/0062426 A1
(43) Pub. Date: Feb. 20, 2025

(54) APPARATUS, SYSTEM AND METHOD FOR DETECTING FAULT OF ALL SOLID STATE BATTERY

(71) Applicants: Hyundai Motor Company, Seoul (KR); Kia Corporation, Seoul (KR)

(72) Inventors: Ga Young Choi, Busan (KR); Ji Chang Kim, Seoul (KR); Gyeong Min Bak, Suwon (KR); Sung Hoo Jung, Changwon (KR); Hyun Min Seo, Seoul (KR)

(21) Appl. No.: 18/604,660

(22) Filed: Mar. 14, 2024

(30) Foreign Application Priority Data
Aug. 17, 2023 (KR) 10-2023-0107850

(51) Int. Cl. H01M 10/48; H01M 10/44

(52) U.S. Cl. CPC H01M 10/48; H01M 10/44

(57) ABSTRACT

The present disclosure relates to an all-solid-state battery defect detection apparatus, system, and method. An exemplary embodiment of the present disclosure provides an all-solid-state battery defect detection apparatus including: a processor configured to detect a defect in an all-solid-state battery by using a pressure distribution measurement of the all-solid-state battery at ends of charging and discharging of the all-solid-state battery immediately after fastening a pressurizing jig to the all-solid-state battery; and a storage configured to store data and algorithms driven by the processor.

### CROSS-REFERENCE TO RELATED APPLICATION

This application claims under 35 U.S.C. § 119(a) the benefit of Korean Patent Application No. 10-2023-0107850, filed in the Korean Intellectual Property Office on Aug. 17, 2023, the entire contents of which are incorporated herein by reference.

### BACKGROUND

### Technical Field

The present disclosure relates to an all-solid-state battery defect detection apparatus, system, and method, and more specifically, to an all-solid-state battery defect detection technology using pressure distribution.

### Background

A secondary battery is a battery capable of repeatedly charging and discharging, and is widely used in high-tech devices such as mobile phones, laptops, and electric vehicles. Recently, a demand for a secondary battery with high energy density and high output is increasing, and an all-solid-state battery is attracting attention as a next-generation technology.

An all-solid-state battery, which is a battery that uses a solid electrolyte between positive and negative electrode layers, may increase energy density, reduce volume, and greatly improve stability by using a solid-state electrolyte compared to conventional lithium-ion batteries.

Due to such characteristics of the all-solid-state battery, high and uniform pressure must be continuously applied using a pressurizing jig. In this case, if the pressurizing jig is tightened unevenly, uneven pressure may be applied to the all-solid-state battery, causing lithium metal to be unevenly deposited or desorbed, and a volume of certain parts to expand or contract, and a part where such expansion or contraction occurs may quickly deteriorate, thereby having negative impact on quality (safety, durability, etc.) of the all-solid-state battery.

### SUMMARY

An exemplary embodiment of the present disclosure attempts to provide an all-solid-state battery defect detection apparatus, system, and method, capable of accurately detecting a defect in an all-solid-state battery using a pressure distribution.

In addition, an exemplary embodiment of the present disclosure attempts to provide an all-solid-state battery defect detection apparatus, system, and method, capable of performing accurate defect detection on an all-solid-state battery by detecting a defect in the all-solid-state battery measuring a pressure distribution at an end of charging of the all-solid-state battery and at an end of discharging of the all-solid-state battery immediately after fastening a pressurizing jig of the all-solid-state battery.

The technical objects of the present disclosure are not limited to the objects mentioned above, and other technical objects not mentioned may be clearly understood by those skilled in the art from the description of the claims.

An exemplary embodiment of the present disclosure provides an all-solid-state battery defect detection apparatus including: a processor configured to detect a defect in an all-solid-state battery by using a pressure distribution measurement of the all-solid-state battery at ends of charging and discharging of the all-solid-state battery immediately after fastening a pressurizing jig to the all-solid-state battery; and a storage configured to store data and algorithms driven by the processor.

In an exemplary embodiment of the present disclosure, the processor may be configured to detect a defect in the all-solid-state battery by comparing a pressure distribution standard deviation value measured immediately after fastening the pressurizing jig to the all-solid-state battery, with a predetermined reference value.

In an exemplary embodiment of the present disclosure, the processor may be configured to detect a defect in the all-solid-state battery by comparing a pressure distribution standard deviation value measured at the end of charging of the all-solid-state battery with a predetermined reference value.

In an exemplary embodiment of the present disclosure, the end of charging the all-solid-state battery may refer to a state in which the charging of the all-solid-state battery begins and a state of charge (SOC) of the all-solid-state battery reaches a predetermined reference value.

In an exemplary embodiment of the present disclosure, the processor may be configured to detect a defect in the all-solid-state battery by comparing a pressure distribution standard deviation value measured at the end of discharging of the all-solid-state battery with a predetermined reference value.

In an exemplary embodiment of the present disclosure, the end of discharging the all-solid-state battery may refer to a state in which the discharging of the all-solid-state battery begins and a depth of discharge (DOD) reaches a predetermined reference value.

In an exemplary embodiment of the present disclosure, the processor may be configured to determine the predetermined reference value according to an amount of pressure applied to a battery cell of the all-solid-state battery and a size of the battery cell.

An exemplary embodiment of the present disclosure provides an all-solid-state battery defect detection system including: a surface pressure sensor configured to measure a pressure distribution of an all-solid-state battery; and an all-solid-state battery defect detection apparatus configured to detect a defect in an all-solid-state battery by using a pressure distribution measurement of the all-solid-state battery measured by the surface pressure sensor at ends of charging and discharging of the all-solid-state battery immediately after fastening a pressurizing jig to the all-solid-state battery.

In an exemplary embodiment of the present disclosure, the surface pressure sensor may be configured to measure a pressure distribution of a positive electrode of the all-solid-state battery.

In an exemplary embodiment of the present disclosure, the surface pressure sensor may be configured to be provided between the pressurization jig and the all-solid-state battery.

In an exemplary embodiment of the present disclosure, the all-solid-state battery defect detection apparatus may be configured to detect a defect in the all-solid-state battery by comparing a pressure distribution standard deviation value measured immediately after fastening the pressurizing jig to the all-solid-state battery, with a predetermined reference value.

In an exemplary embodiment of the present disclosure, the all-solid-state battery defect detection apparatus may be configured to detect a defect in the all-solid-state battery by comparing a pressure distribution standard deviation value measured at the end of charging of the all-solid-state battery with a predetermined reference value.

In an exemplary embodiment of the present disclosure, the all-solid-state battery defect detection apparatus may be configured to detect a defect in the all-solid-state battery by comparing a pressure distribution standard deviation value measured at the end of discharging of the all-solid-state battery with a predetermined reference value.

An exemplary embodiment of the present disclosure provides an all-solid-state battery defect detection method including: detecting a defect in an all-solid-state battery using a pressure distribution measurement of the all-solid-state battery immediately after fastening a pressurizing jig to the all-solid-state battery; detecting a defect in the all-solid-state battery using a pressure distribution measurement of the all-solid-state battery at an end of charging the all-solid-state battery; and detecting a defect in the all-solid-state battery using a pressure distribution measurement of the all-solid-state battery at an end of discharging the all-solid-state battery.

In an exemplary embodiment of the present disclosure, the detecting of the defect in the all-solid-state battery using the pressure distribution measurement of the all-solid-state battery immediately after fastening the pressurizing jig to the all-solid-state, may include: comparing a pressure distribution standard deviation value measured immediately after fastening the pressurizing jig to the all-solid-state battery, with a predetermined reference value; and determining that the all-solid-state battery is defective in response to a case where the pressure distribution standard deviation value measured immediately after fastening the pressurizing jig to the all-solid-state battery is smaller than the predetermined reference value.

In an exemplary embodiment of the present disclosure, the detecting of the defect in the all-solid-state battery using the pressure distribution measurement of the all-solid-state battery at the end of charging the all-solid-state battery, may include: comparing the pressure distribution standard deviation value measured at the end of charging of the all-solid-state battery, with a predetermined reference value; and determining that the all-solid-state battery is defective in response to a case where the pressure distribution standard deviation value measured at the end of charging of the all-solid-state battery is smaller than the predetermined reference value.

In an exemplary embodiment of the present disclosure, the detecting of the defect in the all-solid-state battery using the pressure distribution measurement of the all-solid-state battery at the end of discharging the all-solid-state battery, may include:
comparing the pressure distribution standard deviation value measured at the end of discharging of the all-solid-state battery, with a predetermined reference value; and determining that the all-solid-state battery is defective in response to a case where the pressure distribution standard deviation value measured at the end of discharging of the all-solid-state battery is smaller than the predetermined reference value.

In an exemplary embodiment of the present disclosure, the end of charging the all-solid-state battery may refer to a state in which the charging of the all-solid-state battery begins and a state of charge (SOC) of the all-solid-state battery reaches a predetermined reference value.

In an exemplary embodiment of the present disclosure, the end of discharging the all-solid-state battery may refer to a state in which the discharging of the all-solid-state battery begins and a depth of discharge (DOD) reaches a predetermined reference value.

In an exemplary embodiment of the present disclosure, it may further include determining the predetermined reference value according to an amount of pressure applied to a battery cell of the all-solid-state battery and a size of the battery cell.

According to the present technology, it is possible to accurately detect a defect in an all-solid-state battery using a pressure distribution.

According to the present technology, it is possible to perform accurate defect detection on an all-solid-state battery by detecting a defect in the all-solid-state battery measuring a pressure distribution each at an end of charging of the all-solid-state battery and at an end of discharging of the all-solid-state battery immediately after fastening a pressurizing jig of the all-solid-state battery.

In some embodiments, an all-solid-state battery defect detection apparatus. The apparatus includes a processor configured to detect a defect in an all-solid-state battery by: measuring a pressure distribution of the all-solid-state battery; determining a pressure distribution standard deviation value based on the measured pressure distribution; and comparing the pressure distribution standard deviation value with a predetermined reference value. The apparatus further includes a storage configured to store data and algorithms driven by the processor.

The pressure distribution standard deviation value determined based on the pressure distribution measured immediately after fastening a pressurizing jig to the all-solid-state battery may be compared with the predetermined reference value.

The pressure distribution standard deviation value determined based on the pressure distribution measured during a charging period where a state of charge (SOC) of the all-solid-state battery increases from about 50% to about 100% may be compared with the predetermined reference value.

The pressure distribution standard deviation value determined based on the pressure distribution measured during a discharging period where a state of charge (SOC) of the all-solid-state battery decreases from about 50% to about 0% may be compared with a predetermined reference value.

The processor may be configured to determine the predetermined reference value based on an amount of pressure applied to a battery cell of the all-solid-state battery and a size of the battery cell.

The processor may be configured to determine the predetermined reference value based on an amount of pressure applied to a battery cell of the all-solid-state battery and a size of the battery cell.

The processor may be configured to determine the predetermined reference value based on an amount of pressure applied to a battery cell of the all-solid-state battery and a size of the battery cell.

In some embodiments, an all-solid-state battery defect detection system. The system includes a surface pressure sensor configured to measure a pressure distribution of an all-solid-state battery; and an all-solid-state battery defect detection apparatus configured to detect a defect in an all-solid-state battery by: measuring a pressure distribution of the all-solid-state battery; determining a pressure distribution standard deviation value based on the measured pressure distribution; and comparing the pressure distribution standard deviation value with a predetermined reference value.

The surface pressure sensor may be configured to measure a pressure distribution of a positive electrode of the all-solid-state battery.

The surface pressure sensor may be configured to be provided between a pressurization jig and the all-solid-state battery.

The all-solid-state battery defect detection apparatus may be configured to detect a defect in the all-solid-state battery by comparing the pressure distribution standard deviation value determined based on the pressure distribution measured immediately after fastening a pressurizing jig to the all-solid-state battery, with a predetermined reference value.

The all-solid-state battery defect detection apparatus may be configured to detect a defect in the all-solid-state battery by comparing the pressure distribution standard deviation value determined based on the pressure distribution measured during a charging period where a state of charge (SOC) of the all-solid-state battery increases from about 50% to about 100%, with a predetermined reference value.

The all-solid-state battery defect detection apparatus may be configured to detect a defect in the all-solid-state battery by comparing the pressure distribution standard deviation value determined based on the pressure distribution measured during a discharging period where a state of charge (SOC) of the all-solid-state battery decreases from about 50% to about 0%, with a predetermined reference value.

In some embodiments, an all-solid-state battery defect detection method is provided. The method comprises measuring a pressure distribution of the all-solid-state battery; determining a pressure distribution standard deviation value based on the measured pressure distribution; and comparing the pressure distribution standard deviation value with a predetermined reference value.

The pressure distribution standard deviation value determined based on the pressure distribution measured immediately after fastening a pressurizing jig to the all-solid-state battery may be compared with the predetermined reference value.

The pressure distribution standard deviation value determined based on the pressure distribution measured during a charging period where a state of charge (SOC) of the all-solid-state battery increases from about 50% to about 100% may be compared with the predetermined reference value.

The pressure distribution standard deviation value determined based on the pressure distribution measured during a discharging period where a state of charge (SOC) of the all-solid-state battery decreases from about 50% to about 0% may be compared with the predetermined reference value.

The method may further comprise determining that the all-solid-state battery is defective if the pressure distribution standard deviation value is smaller than the predetermined reference value.

The method may further comprise determining that the all-solid-state battery is defective if the pressure distribution standard deviation value is smaller than the predetermined reference value.

The method may further comprise determining that the all-solid-state battery is defective if the pressure distribution standard deviation value is smaller than the predetermined reference value.

Furthermore, various effects that can be directly or indirectly identified through this document may be provided.

As discussed, the method and system suitably include use of a controller or processer.

### BRIEF DESCRIPTION OF THE DRAWINGS

FIG. 1 illustrates a block diagram showing an example configuration of an all-solid-state defect detection system including an all-solid-state defect detection apparatus.

FIG. 2 illustrates a view for describing an example pressure measurement point for detecting a defect in an all-solid-state battery.

FIG. 3 illustrates an example pressure distribution for each pressure measurement point for detecting a defect in an all-solid-state battery.

FIG. 4 illustrates an example pressure distribution for each battery SOC.

FIG. 5 illustrates a schematic view showing example pressurization of an all-solid-state battery.

FIG. 6 illustrates a flowchart showing an example an all-solid-state battery defect detection method.

FIG. 7A and FIG. 7B each illustrate a view for describing an example lifespan of a battery cell in detecting a defect using a pressure distribution.

FIG. 8 illustrates an example computing system.

### DETAILED DESCRIPTION

Hereinafter, some exemplary embodiments of the present disclosure will be described in detail with reference to exemplary drawings. It should be noted that in adding reference numerals to constituent elements of each drawing, the same constituent elements have the same reference numerals as possible even though they are indicated on different drawings. In describing an exemplary embodiment, when it is determined that a detailed description of the well-known configuration or function associated with the exemplary embodiment may obscure the gist of the present disclosure, it will be omitted.

In describing constituent elements according to an exemplary embodiment, terms such as first, second, A, B, (a), and (b) may be used. These terms are only for distinguishing the constituent elements from other constituent elements, and the nature, sequences, or orders of the constituent elements are not limited by the terms. Furthermore, all terms used herein including technical scientific terms have the same meanings as those which are generally understood by those skilled in the technical field to which an exemplary embodiment of the present disclosure pertains (those skilled in the art) unless they are differently defined. Terms defined in a generally used dictionary shall be construed to have meanings matching those in the context of a related art, and shall not be construed to have idealized or excessively formal meanings unless they are clearly defined in the present specification.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

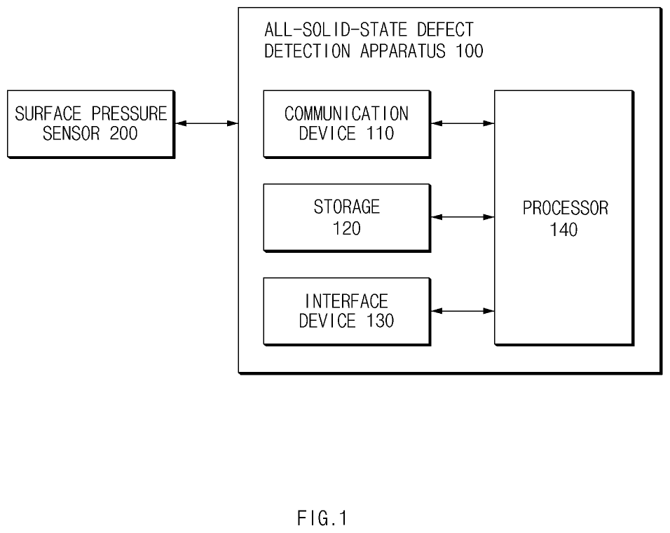

Hereinafter, various exemplary embodiments of the present disclosure will be described in detail with reference to FIG. 1 to FIG. 8.

FIG. 1 illustrates a block diagram showing an example configuration of an all-solid-state defect detection system including an all-solid-state defect detection apparatus.

Referring to FIG. 1, the all-solid-state defect detection system according to an exemplary embodiment of the present disclosure may include an all-solid-state battery defect detection apparatus 100 and a surface pressure sensor 200.

The all-solid-state battery defect detection apparatus 100 according to the present disclosure may be formed integrally with an all-solid-state battery, may be implemented as a separate hardware device to be connected to the all-solid-state battery by a connection means, and may also be implemented as installed and attached to the all-solid-state battery.

The all-solid-state battery defect detection apparatus 100 may detect a defect in an all-solid-state battery by obtaining a pressure distribution of the all-solid-state battery at an end of charging of the all-solid-state battery and at an end of discharging of the all-solid-state battery immediately after fastening a pressurizing jig. In this case, immediately after fastening the pressurizing jig indicate in which the pressurizing jig has been fastened but no pressure has yet been applied. The end of charging refers to a time when the charging of the all-solid-state battery begins and the charging is almost completed, and refers to a section where a state of charge (SOC) of the all-solid-state battery increases from 50% to 100%. The end of discharging refers to a section where a depth of discharge (DOD) of the all-solid-state battery decreases from 50% to 0% as a time point where the discharging is almost completed.

The all-solid-state battery defect detection apparatus 100 may include a communication device 110, a storage 120, an interface device 130, and a processor 140. According to an exemplary embodiment of the present disclosure, the for the all-solid-state battery defect detection apparatus 100 may be implemented as a single unit by coupling components with each other, and some components may be omitted.

The communication device 110 may be a hardware device implemented with various electronic circuits to transmit and receive signals through a wireless or wired connection, and may receive a sensing result by communicating with the surface pressure sensor 200, etc.

In addition, the communication device 110 may transmit a defect detection result of the all-solid-state battery to an external server, etc. as a result of the pressure distribution of the all-solid-state battery by communicating with an external server through a mobile communication technique, a wireless Internet access, or a short range communication technique,

Herein, the wireless Internet communication may include wireless LAN (WLAN), wireless-fidelity (Wi-Fi), Wi-Fi direct, digital living network alliance (DLNA), wireless broadband (WiBro), world interoperability for microwave access (WiMAX), high speed downlink packet access (HSDPA), high speed uplink packet access (HSUPA), long term evolution (LTE), long term evolution-advanced (LTE-A), or the like. In addition, short-range communication technique may include Bluetooth, ZigBee, ultra-wideband (UWB), radio frequency identification (RFID), infrared data association (IrDA), and the like.

The mobile communication technique may include technical standards, communication methods for mobile communication (e.g., global system for mobile communication (GSM), code division multi access (CDMA), code division multi access 2000 (CDMA 2000), enhanced voice-data optimized or enhanced voice-data only (EV-DO), wideband CDMA (WCDMA), high speed downlink packet access (HSDPA), high speed uplink packet access (HSUPA), long term evolution (LTE), long term evolution-advanced (LTE-A), 4th generation mobile telecommunication (4G), 5th generation mobile telecommunication (5G)), or the like.

The storage 120 may store sensing results of the surface pressure sensor 200 and data and/or algorithms required for the processor 140 to operate, and the like.

As an example, the storage 120 may store a reference value for comparison with a standard deviation of the pressure distribution. This reference value may be predetermined and stored based on experimental values.

The storage 120 may include a storage medium of at least one type among memories of types such as a flash memory, a hard disk, a micro, a card (e.g., a secure digital (SD) card or an extreme digital (XD) card), a random access memory (RAM), a static RAM (SRAM), a read-only memory (ROM), a programmable ROM (PROM), an electrically erasable PROM (EEPROM), a magnetic memory (MRAM), a magnetic disk, and an optical disk.

The interface device 130 may include an input means for receiving a control command from a user and an output means for outputting an operation state of the apparatus 100 and results thereof. Herein, the input means may include a key button, and may include a mouse, a joystick, a jog shuttle, a stylus pen, and the like. Furthermore, the input means may include a soft key implemented on the display.

The output device may include a display, and may also include a voice output means such as a speaker. In the instant case, in a response to a case that a touch sensor formed of a touch film, a touch sheet, or a touch pad is provided on the display, the display may operate as a touch screen, and may be implemented in a form in which an input device and an output device are integrated. In the present disclosure, the output means may output a defect detection result of an all-solid-state battery, etc.

In the instant case, the display may include at least one of a liquid crystal display (LCD), a thin film transistor liquid crystal display (TFT LCD), an organic light emitting diode display (OLED display), a flexible display, a field emission display (FED), a 3D display, or any combination thereof.

The processor 140 may be electrically connected to the communication device 110, the storage 120, the interface device 130, and the like, may electrically control each component, and may be an electrical circuit that executes software commands, thereby performing various data processing and calculations described below.

The processor 140 may perform overall control such that each component can normally perform their functions by processing a signal transferred between each component of the all-solid-state battery defect detection apparatus 100. The processor 140 may be implemented in the form of hardware, software, or a combination of and software. For example, the processor 140 may be implemented as a microprocessor, but the present disclosure is not limited thereto.

The processor 140 may detect a defect in the all-solid-state battery by using a pressure distribution measurement of the all-solid-state battery at ends of charging and discharging of the all-solid-state battery immediately after fastening the pressurizing jig to the all-solid-state battery.

In order to continuously apply uniform pressure to the all-solid-state battery, the processor 140 may detect a defect in the all-solid-state battery by comparing a measured pressure distribution standard deviation value with a predetermined reference value immediately after fastening the pressurizing jig, i.e. before starting pressurization.

That is, the processor 140 may compare the pressure distribution standard deviation value measured immediately after fastening the pressurizing jig to the all-solid-state battery with the predetermined reference value, and may determine that the all-solid-state battery is defective in response to a case where the pressure distribution standard deviation value measured at the end of charging of the all-solid-state battery is smaller than the predetermined reference value.

The processor 140 may detect a defect in the all-solid-state battery by comparing the pressure distribution standard deviation value measured at the end of charging of the all-solid-state battery with the predetermined reference value.

That is, the processor 140 may compare the pressure distribution standard deviation value measured immediately at the end of charging of the all-solid-state battery with the predetermined reference value, and may determine that the all-solid-state battery is defective in response to a case where the pressure distribution standard deviation value measured at the end of charging of the all-solid-state battery is smaller than the predetermined reference value.

The processor 140 may detect a defect in the all-solid-state battery by comparing the pressure distribution standard deviation value measured at the end of discharging of the all-solid-state battery with the predetermined reference value.

That is, the processor 140 may compare the pressure distribution standard deviation value measured immediately at the end of discharging of the all-solid-state battery with the predetermined reference value, and may determine that the all-solid-state battery is defective in response to a case where the pressure distribution standard deviation value measured at the end of discharging of the all-solid-state battery is smaller than the predetermined reference value.

In this case, the end of charging of the all-solid-state battery refers to a state in which the charging of the all-solid-state battery begins and the state of charge (SOC) of the all-solid-state battery reaches a predetermined reference value. In addition, the end of discharging of the all-solid-state battery refers to a state in which the discharging of the all-solid-state battery begins and the depth of discharge (DOD) reaches a predetermined reference value.

The processor 140 may determine the predetermined reference value according to an amount of pressure applied to a battery cell of the all-solid-state battery and a size of the battery cell.

The surface pressure sensor 200 may measure a pressure distribution of a positive or negative electrode of the all-solid-state battery. The present disclosure discloses an example of measuring the pressure distribution of the positive electrode of the all-solid-state battery, but it is not limited thereto.

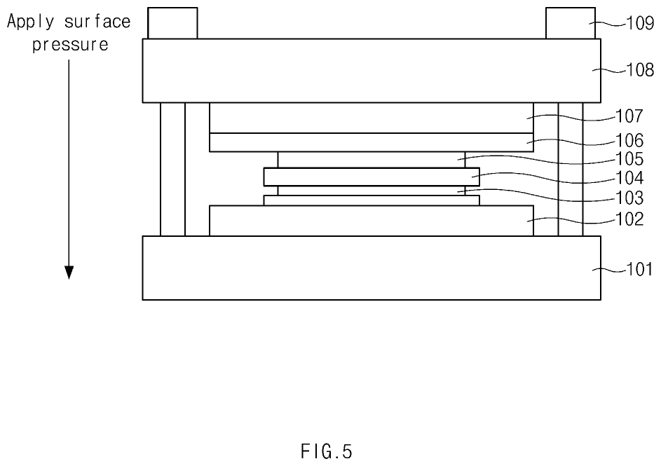

For this purpose, a surface pressure sensor may be provided at an upper portion of the positive electrode of the all-solid-state battery, and will be described in detail later with reference to FIG. 5.

As such, according to the present disclosure, it is possible to detect a defect in the all-solid-state battery using the pressure distribution of the all-solid-state battery. However, a common technique for detecting a defect in the all-solid-state battery is to use open circuit voltage (OCV), and a method for detecting a defect in the all-solid-state battery using open circuit voltage and a method for detecting a defect in the all-solid-state battery using the pressure distribution according to the present disclosure may be used together to detect a defect in the all-solid-state battery, thereby increasing defect detection accuracy.

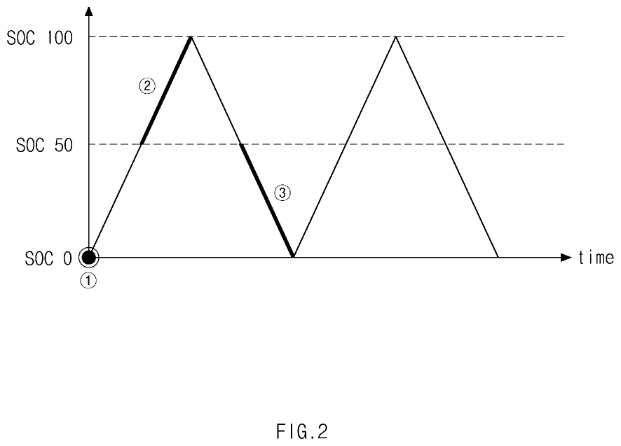

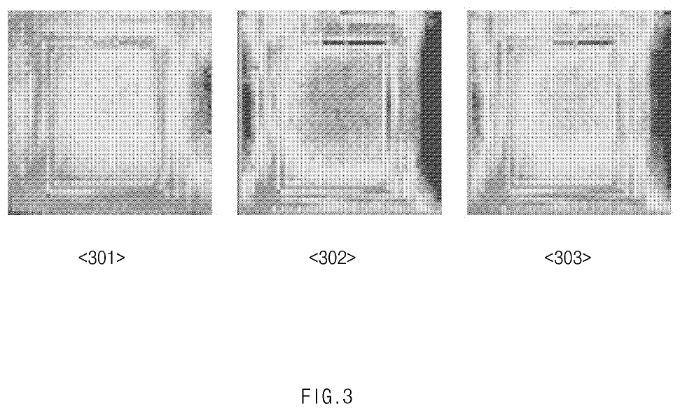

FIG. 2 illustrates a view for describing an example pressure measurement point for detecting a defect in an all-solid-state battery, and FIG. 3 illustrates an example pressure distribution for each pressure measurement point for detecting a defect in an all-solid-state battery.

Referring to FIG. 2, in the present disclosure, the pressure of the all-solid-state battery may be measured \textcircled{1} immediately after fastening the pressurizing jig, \textcircled{2} at the end of charging the all-solid-state battery, and \textcircled{3} at the end of discharging the all-solid-state battery.

In response to a case where a defect in the all-solid-state battery is detected immediately after fastening the pressurized jig, defect screening may be performed even without applying current to battery cells, thereby increasing shipment of good battery cells per hour. In addition, activation of charging and discharging takes a long time, defect detection efficiency may be increased by detecting a defect in the all-solid-state battery in advance using the pressure distribution before the activation. Accordingly, the pressure to detect a defect in the all-solid-state battery is measured immediately after fastening the pressurizing jig. A view 301 of FIG. 3 shows the pressure distribution of the all-solid-state battery immediately after fastening the pressurizing jig.

In addition, oxidative decomposition of an electrolyte actively occurs at a positive electrode interface and reductive decomposition of an electrolyte occurs at a negative electrode interface at the end of charging of the all-solid-state battery, so side reactants are generated at each of the interfaces. A degree of this reaction varies depending on the resistance of the interfaces, a difference in pressure distribution due to volume contraction and expansion at spots where a main reaction and a side reaction occur is significant. Accordingly, it is necessary to detect a defect in the all-solid-state battery by measuring the pressure at the end of charging the all-solid-state battery. In this case, the main reaction at the end of charging of the all-solid-state battery may include deintercalation of lithium (Li) from the positive electrode and intercalation of lithium (Li) from the negative electrode. A view 302 of FIG. 3 discloses the pressure distribution of the all-solid-state battery in response to a case where the SOC is 100, i.e., at the end of charging.

In addition, reductive decomposition of the electrolyte may occur at the positive electrode interface, and oxidative decomposition of the electrolyte may occur at the negative electrode interface at the end of discharging of the all-solid-state battery, generating different side reactants from those at the end of charging. Likewise, a difference in pressure distribution that was not visible at the end of charging appears significant. In this case, the main reaction at the end of charging may include intercalation of lithium (Li) from the positive electrode and deintercalation of lithium (Li) from the negative electrode. A view 303 of FIG. 3 discloses the pressure distribution of the all-solid-state battery in response to a case where the depth of discharge (DOD) is 20, i.e., at the end of discharging.

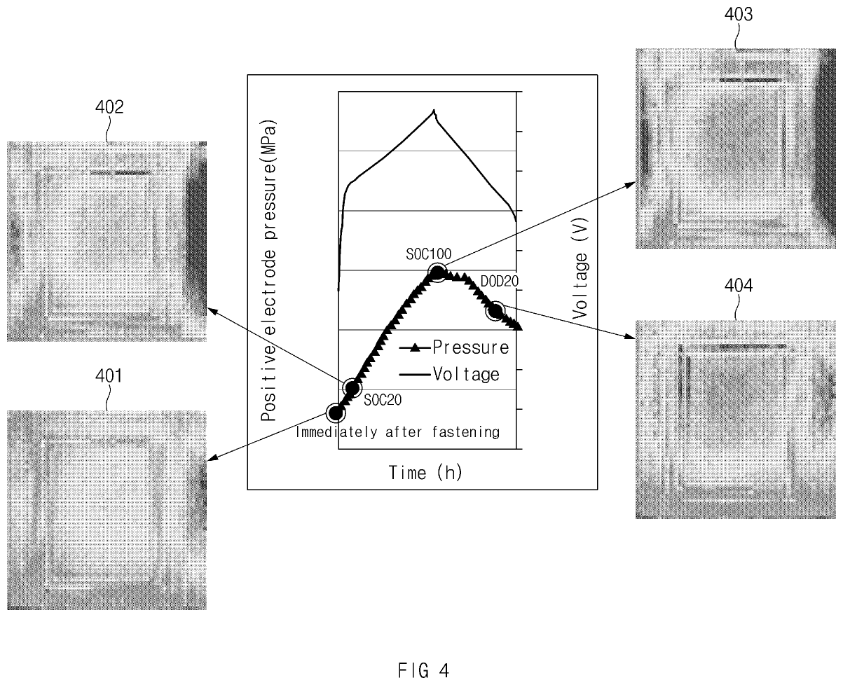

FIG. 4 illustrates an example pressure distribution for each battery SOC.

Referring to FIG. 4, it may be seen that a physical pressure distribution in which no electrochemical reaction occurs, as shown in a view 402, immediately after fastening the pressurizing jig. In a SOC (0≤x≤50), even if an electrochemical reaction proceeds, a degree of volume expansion or contraction is relatively not severe. A view 402 shows the pressure distribution in response to a case where the SOC is 20.

In the SOC (50≤x≤100), components that configure the positive electrode may be exposed to a relatively high potential, and components that configures the negative electrode may be exposed to a relatively low potential. Accordingly, electrochemical reactions occur actively within each electrode or at interfaces thereof, resulting in volume expansion and contraction, making it easy to determine the pressure distribution.

Additionally, since the volume expansion of a cell is the largest at the SOC of 100, it is easiest to observe the resulting pressure distribution. A view 403 shows the pressure distribution in response to a case where the SOC is 100.

Similarly, even in the DOD (0≤y≤50), components that configure a cell are exposed to a relatively low potential state, so an electrochemical reaction that occurs at a low voltage is active, making it easy to determine the pressure distribution. A view 404 shows the pressure distribution in response to a case where the DOD is 20.

The reference value may be set differently depending on a magnitude of a force applied to a battery cell of the all-solid-state battery and a size of the battery cell.

FIG. 5 illustrates a schematic view showing example pressurization of an all-solid-state battery.

Referring to FIG. 5, for a surface pressure sensor, a silicon pad 102 is positioned on the pressurizing jig 101, an all-solid-state battery is positioned by sequentially stacking a negative electrode 103, a solid electrolyte 104, and a positive electrode 105 on the silicon pad 102, a surface pressure sensor 106 is positioned at an upper portion of the all-solid-state battery, a silicon pad 107 is positioned on the surface pressure sensor 106, and a pressurizing jig 108 is positioned at an upper portion of the silicon pad 107, and bolts 109 for fastening the pressurizing jigs 101 and 108 are formed. FIG. 5 illustrates a state in which the pressuring jigs 101 and 108 are fastened to the all-solid-state battery.

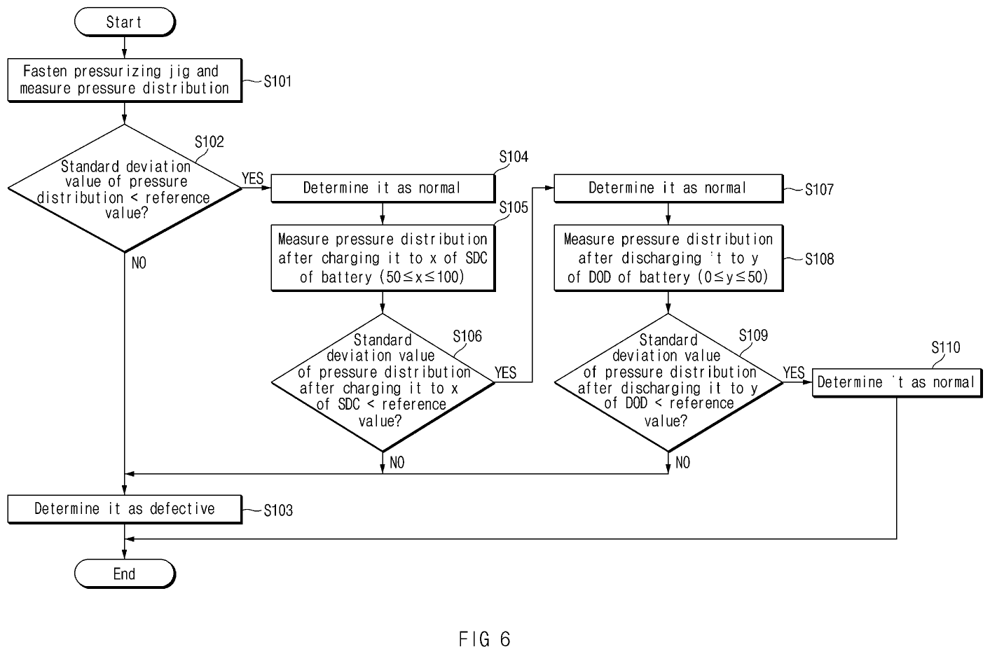

Hereinafter, an all-solid-state battery defect detection method according to an exemplary embodiment of the present disclosure will be described with reference to FIG. 6. FIG. 6 illustrates a flowchart showing an example an all-solid-state battery defect detection method.

Hereinafter, it is assumed that the all-solid-state battery defect detection apparatus 100 of FIG. 1 performs processes of FIG. 6. In addition, in the description of FIG. 6, operations described as being performed by a device may be understood as being controlled by the processor 140 of the all-solid-state battery defect detection apparatus 100. In following exemplary embodiments, operations of steps S101 to S110 may be performed sequentially, but are not necessarily performed sequentially. For example, an order of each operation may be changed, and at least two operations may be performed in parallel.

Referring to FIG. 6, the all-solid-state battery defect detection apparatus 100 measures the pressure distribution of the positive electrode of the all-solid-state battery by using the surface pressure sensor 106 immediately after fastening the pressurizing jigs 101 and 108 to the all-solid-state battery (S101).

Accordingly, the all-solid-state battery defect detection apparatus 100 may determine a standard deviation of the pressure distribution immediately after fastening the pressurizing jigs 101 and 108. In this case, the all-solid-state battery may include a plurality of battery cells, and the all-solid-state battery defect detection apparatus 100 may determine the standard deviation of the pressure distribution immediately after fastening the pressurizing jigs 101 and 108 of a predetermined number of battery cells (e.g., 50) by obtaining the pressure distribution immediately after fastening the pressurizing jigs 101 and 108 of the predetermined number of battery cells (e.g., 50).

The all-solid-state battery defect detection apparatus 100 may determine whether the standard deviation of the pressure distribution is smaller than a predetermined reference value immediately after the pressure jigs 101 and 108 are fastened (S102). In this case, the, the reference value may be pre-determined by an experimental value to be stored in the storage 120.

In response to a case where the standard deviation of the pressure distribution immediately after fastening the pressurizing jigs 101 and 108 is greater than or equal to the predetermined reference value, the all-solid-state battery defect detection apparatus 100 may determine that corresponding battery cells (e.g., 50 battery cells subject to standard deviation) are defective (S103).

On the other hand, in response to a case where the standard deviation of the pressure distribution immediately after fastening the pressurizing jigs 101 and 108 is smaller than the predetermined reference value, the all-solid-state battery defect detection apparatus 100 may determine that corresponding battery cells (e.g., 50 battery cells subject to standard deviation) are normal (S104).

Next, the all-solid-state battery defect detection apparatus 100 may measure the pressure distribution and determine the standard deviation of the pressure distribution by using the surface pressure sensor 106 at the end of charging after charging it to x of the SOC of the all-solid-state battery (S105).

The all-solid-state battery defect detection apparatus 100 may determine whether the standard deviation of the pressure distribution measured after charging it to x of the SOC of the all-solid-state battery is smaller than a predetermined reference value (S106).

Accordingly, the all-solid-state battery defect detection apparatus 100 may determine that the all-solid-state battery is normal in response to a case where the standard deviation of the pressure distribution measured after charging it to x (50≤x≤100) of the SOC of the all-solid-state battery is smaller than the predetermined reference value (S107), and may determine that the solid-state battery is defective in response to a case where the standard deviation of the pressure distribution measured after charging it to x of the SOC of the all-solid-state battery is equal to or greater than the predetermined reference value (S103).

Next, the all-solid-state battery defect detection apparatus 100 may measure the pressure distribution of the all-solid-state battery after discharging it to y (0≤y≤50) of the DOD of the all-solid-state battery to determine a standard deviation of the pressure distribution (S108).

The all-solid-state battery defect detection apparatus 100 may determine whether a standard deviation value of the pressure distribution measured after discharging it to y (0≤y≤50) of the DOD of the all-solid-state battery is smaller than a predetermined reference value (S109).

Accordingly, the all-solid-state battery defect detection apparatus 100 may determine that the all-solid-state battery is normal in response to a case where the standard deviation of the pressure distribution measured after discharging it to y (0≤y≤50) of the DOD of the all-solid-state battery is smaller than the predetermined reference value (S110), and may determine that the solid-state battery is defective in response to a case where the standard deviation of the pressure distribution measured after discharging it to y (0≤y≤50) of the DOD of the all-solid-state battery is equal to or greater than the predetermined reference value (S103).

As such, according to the present disclosure, accuracy of detecting a defect in the all-solid-state battery may be increased by detecting defects in the all-solid-state battery three times measuring the pressure distribution of the all-solid-state battery at the end of charging and at the end of discharging, immediately after fastening the pressurizing jig.

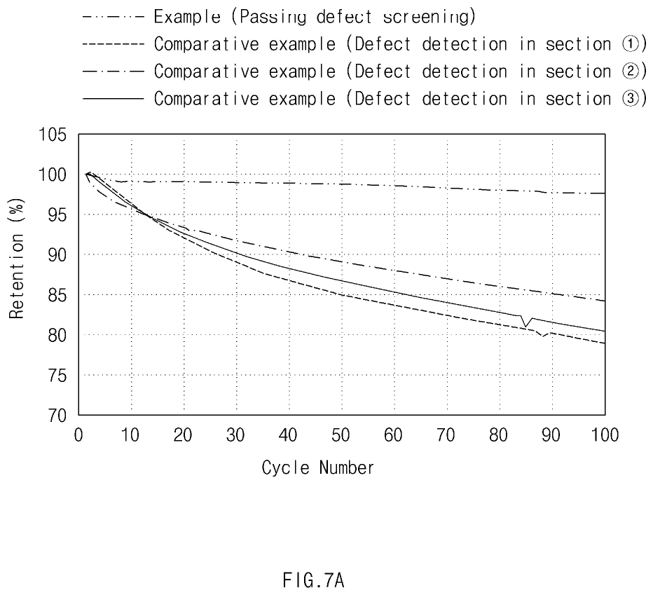

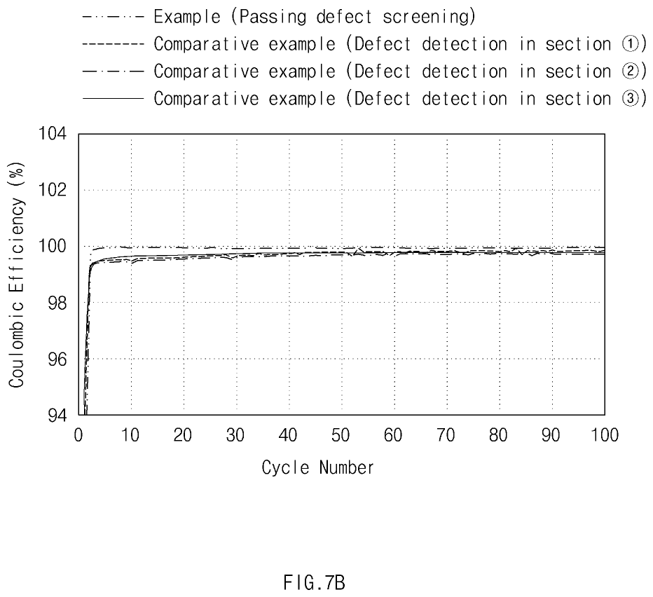

FIG. 7A and FIG. 7B each illustrate a view for describing an example lifespan of a battery cell in detecting a defect using a pressure distribution.

FIG. 7A illustrates retention for each number of cycles. It may be seen that initial retention of battery cells that have passed defect screening, that is, normal battery cells, rarely drops from 100. However, it may be seen that retentions of a battery cell detected as defective in a section \textcircled{1}, a battery cell detected as defective in a section \textcircled{2}, and a battery cell detected as defective in a section \textcircled{3} of FIG. 2 decreases to 80 as the number of cycles increases. This decrease in retention indicates that a lifespan of the battery cell decreases.

FIG. 7B illustrates coulombic efficiency for each number of cycles. It may be seen that initial coulombic efficiency of battery cells that have passed defect screening, that is, normal battery cells, rarely drops from 100. However, it may be seen that coulombic efficiencies of a battery cell detected as defective in a section \textcircled{1}, a battery cell detected as defective in a section \textcircled{2}, and a battery cell detected as defective in a section \textcircled{3} of FIG. 2 decreases as the number of cycles increases. This decrease in coulombic efficiency indicates that a lifespan of the battery cell decreases.

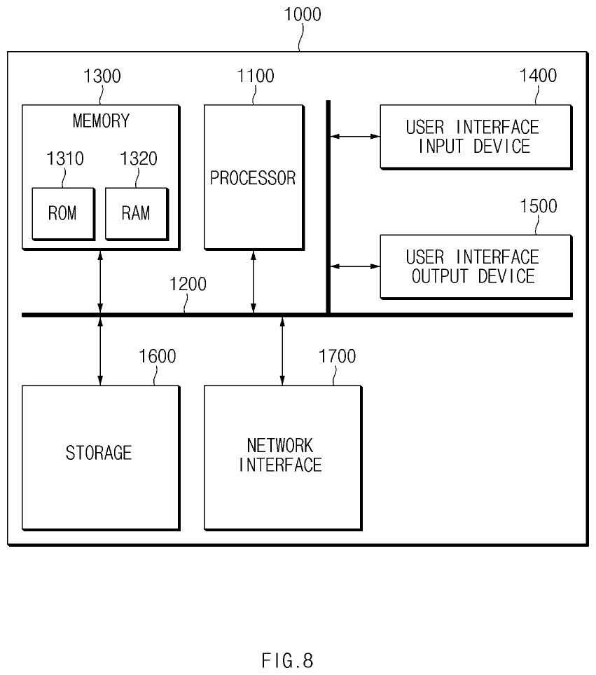

FIG. 8 illustrates an example computing system.

Referring to FIG. 8, the computing system 1000 includes at least one processor 1100 connected through a bus 1200, a memory 1300, a user interface input device 1400, a user interface output device 1500, and a storage 1600, and a network interface 1700.

The processor 1100 may be a central processing unit (CPU) or a semiconductor device that performs processing on commands stored in the memory 1300 and/or the storage 1600. The memory 1300 and the storage 1600 may include various types of volatile or nonvolatile storage media. For example, the memory 1300 may include a read only memory (ROM) 1310 and a random access memory (RAM) 1320.

Accordingly, steps of a method or algorithm described in connection with the exemplary embodiments disclosed herein may be directly implemented by hardware, a software module, or a combination of the two, executed by the processor 1100. The software module may reside in a storage medium (i.e., the memory 1300 and/or the storage 1600) such as a RAM memory, a flash memory, a ROM memory, an EPROM memory, an EEPROM memory, a register, a hard disk, a removable disk, and a CD-ROM.

An exemplary storage medium is coupled to the processor 1100, which can read information from and write information to the storage medium. Alternatively, the storage medium may be integrated with the processor 1100. The processor and the storage medium may reside within an application specific integrated circuit (ASIC). The ASIC may reside within a user terminal. Alternatively, the processor and the storage medium may reside as separate components within the user terminal.

The above description is merely illustrative of the technical idea of the present disclosure, and those skilled in the art to which the present disclosure pertains may make various modifications and variations without departing from the essential characteristics of the present disclosure.

Therefore, the exemplary embodiments disclosed in the present disclosure are not intended to limit the technical ideas of the present disclosure, but to explain them, and the scope of the technical ideas of the present disclosure is not limited by these exemplary embodiments. The protection range of the present disclosure should be interpreted by the claims below, and all technical ideas within the equivalent range should be interpreted as being included in the scope of the present disclosure.

What is claimed is:

1. An all-solid-state battery defect detection apparatus comprising:
a processor configured to detect a defect in an all-solid-state battery by using a pressure distribution measurement of the all-solid-state battery at ends of charging and discharging of the all-solid-state battery immediately after fastening a pressurizing jig to the all-solid-state battery; and
a storage configured to store data and algorithms driven by the processor.

2. The all-solid-state battery defect detection apparatus of claim 1, wherein
the processor is configured
to detect a defect in the all-solid-state battery by comparing a pressure distribution standard deviation value measured immediately after fastening the pressurizing jig to the all-solid-state battery, with a predetermined reference value.

3. The all-solid-state battery defect detection apparatus of claim 1, wherein the processor is configured
to detect a defect in the all-solid-state battery by comparing a pressure distribution standard deviation value measured at the end of charging of the all-solid-state battery with a predetermined reference value.

4. The all-solid-state battery defect detection apparatus of claim 1, wherein
the end of charging the all-solid-state battery
refers to a state in which the charging of the all-solid-state battery begins and a state of charge (SOC) of the all-solid-state battery reaches a predetermined reference value.

5. The all-solid-state battery defect detection apparatus of claim 1, wherein
the processor is configured
to detect a defect in the all-solid-state battery by comparing a pressure distribution standard deviation value measured at the end of discharging of the all-solid-state battery with a predetermined reference value.

6. The all-solid-state battery defect detection apparatus of claim 1, wherein
the end of discharging the all-solid-state battery
refers to a state in which the discharging of the all-solid-state battery begins and a depth of discharge (DOD) reaches a predetermined reference value.

7. The all-solid-state battery defect detection apparatus of claim 2, wherein
the processor is configured
to determine the predetermined reference value according to an amount of pressure applied to a battery cell of the all-solid-state battery and a size of the battery cell.

8. An all-solid-state battery defect detection system comprising:
a surface pressure sensor configured to measure a pressure distribution of an all-solid-state battery; and
an all-solid-state battery defect detection apparatus configured to detect a defect in an all-solid-state battery by using a pressure distribution measurement of the all-solid-state battery measured by the surface pressure sensor at ends of charging and discharging of the all-solid-state battery immediately after fastening a pressurizing jig to the all-solid-state battery.

9. The all-solid-state battery defect detection system of claim 8, wherein
the surface pressure sensor is configured
to measure a pressure distribution of a positive electrode of the all-solid-state battery.

10. The all-solid-state battery defect detection system of claim 8, wherein
the surface pressure sensor is configured
to be provided between the pressurization jig and the all-solid-state battery.

11. The all-solid-state battery defect detection system of claim 8, wherein
the all-solid-state battery defect detection apparatus configured
to detect a defect in the all-solid-state battery by comparing a pressure distribution standard deviation value measured immediately after fastening the pressurizing jig to the all-solid-state battery, with a predetermined reference value.

12. The all-solid-state battery defect detection system of claim 8, wherein
the all-solid-state battery defect detection apparatus configured
to detect a defect in the all-solid-state battery by comparing a pressure distribution standard deviation value measured at the end of charging of the all-solid-state battery with a predetermined reference value.

13. The all-solid-state battery defect detection system of claim 8, wherein
the all-solid-state battery defect detection apparatus configured
to detect a defect in the all-solid-state battery by comparing a pressure distribution standard deviation value measured at the end of discharging of the all-solid-state battery with a predetermined reference value.

14. An all-solid-state battery defect detection method comprising:
detecting a defect in an all-solid-state battery using a pressure distribution measurement of the all-solid-state battery immediately after fastening a pressurizing jig to the all-solid-state battery;
detecting a defect in the all-solid-state battery using a pressure distribution measurement of the all-solid-state battery at an end of charging the all-solid-state battery; and
detecting a defect in the all-solid-state battery using a pressure distribution measurement of the all-solid-state battery at an end of discharging the all-solid-state battery

15. The all-solid-state battery defect detection method of claim 14, wherein
the detecting of the defect in the all-solid-state battery using the pressure distribution measurement of the all-solid-state battery immediately after fastening the pressurizing jig to the all-solid-state, includes:
comparing a pressure distribution standard deviation value measured immediately after fastening the pressurizing jig to the all-solid-state battery, with a predetermined reference value; and
determining that the all-solid-state battery is defective in response to a case where the pressure distribution standard deviation value measured immediately after fastening the pressurizing jig to the all-solid-state battery is smaller than the predetermined reference value.

16. The all-solid-state battery defect detection method of claim 14, wherein
the detecting of the defect in the all-solid-state battery using the pressure distribution measurement of the all-solid-state battery at the end of charging the all-solid-state battery, includes:
comparing the pressure distribution standard deviation value measured at the end of charging of the all-solid-state battery, with a predetermined reference value; and
determining that the all-solid-state battery is defective in response to a case where the pressure distribution standard deviation value measured at the end of charging of the all-solid-state battery is smaller than the predetermined reference value.

17. The all-solid-state battery defect detection method of claim 14, wherein
the detecting of the defect in the all-solid-state battery using the pressure distribution measurement of the all-solid-state battery at the end of discharging the all-solid-state battery, includes:
comparing the pressure distribution standard deviation value measured at the end of discharging of the all-solid-state battery, with a predetermined reference value; and
determining that the all-solid-state battery is defective in response to a case where the pressure distribution standard deviation value measured at the end of discharging of the all-solid-state battery is smaller than the predetermined reference value.

18. The all-solid-state battery defect detection method of claim 14, wherein
the end of charging the all-solid-state battery
refers to a state in which the charging of the all-solid-state battery begins and a state of charge (SOC) of the all-solid-state battery reaches a predetermined reference value.

19. The all-solid-state battery defect detection method of claim 14, wherein
the end of discharging the all-solid-state battery
refers to a state in which the discharging of the all-solid-state battery begins and a depth of discharge (DOD) reaches a predetermined reference value.

20. The all-solid-state battery defect detection method of claim 15, further comprising
determining the predetermined reference value according to an amount of pressure applied to a battery cell of the all-solid-state battery and a size of the battery cell.
